(19) United States
(12) Patent Application Publication
(10) Pub. No.: US 2013/0183360 A1
(43) Pub. Date: Jul. 18, 2013

(54) PETROLATUM COMPOSITION AND PROCESS FOR ITS MANUFACTURE

(71) Applicants: Alexander Lips, Parkgate (GB); Tamara Litvin, Trumbull (GB)

(72) Inventors: Alexander Lips, Parkgate (GB); Tamara Litvin, Trumbull (GB)

(21) Appl. No.: 13/825,411

(22) PCT Filed: Sep. 20, 2011
(86) PCT No.: PCT/EP11/66309

§ 371 Date: Mar. 21, 2013

Related U.S. Application Data
(60) Provisional application No. 61/388,182, filed on Sep. 30, 2010.

(51) Int. Cl. A61K 8/31; A61Q 19/00; A61K 8/02

(52) U.S. Cl. CPC A61K 8/31; A61K 8/02; A61Q 19/00
USPC 424/401

(57) ABSTRACT

Petrolatum based compositions (i.e., composition in which the emollient petrolatum comprises greater than or equal to 50% of the composition) are desirable because of the excellent moisturization properties provided by petrolatum. However, it has sensory negatives perceived by consumers. Specifically, because of the oily/greasy and “tacky” (i.e., slightly adhesive or gummy to touch) feel, petrolatum has certain limits to the scope of application. We have found a way to overcome this problem using a copolymer which furthermore avoids the undesirable use of additional oil. A composition is therefore provided comprising: i) 50% to 99.95% by weight petrolatum; ii) greater than 0.05% by weight of a copolymer bearing greater than 70% long chain alkyl. said composition having an enthalpy of between 30 and 65, preferably 40 and 60, even more preferably 45 and 55 J/g, the petrolatum number average crystal size being less than 10 μm, preferably less than 5 μm, more preferably less than 1 μm.

The present invention relates to a petrolatum composition and a process for its manufacture. It also relates to a cosmetic composition comprising said petrolatum composition.

### Background

Petrolatum based compositions (i.e., composition in which the emollient petrolatum comprises greater than or equal to 50% of the composition) are desirable because of the excellent moisturization properties provided by petrolatum. However, it has sensory negatives perceived by consumers. Specifically, because of the oily/greasy and “tacky” (i.e., slightly adhesive or gummy to touch) feel, petrolatum has certain limits to the scope of application.

We have found a way to overcome this problem using a copolymer which furthermore avoids the undesirable use of additional oil.

Tests and definitions

DSC measurements were performed using Q1000 (TA Instruments, New Castle, Del.) with scanning rate 5 deg/min. The melting enthalpy for the samples approximately 8 mg by weight was obtained in the temperature range −40 to 80 deg C.

Differential scanning calorimetry (DSC) is a widely used technique which was introduced in 1960s. The basic principle underlying this technique is that, when the sample undergoes a physical transformation such as phase transition, more or less heat will need to flow to it than the reference to maintain both at the same temperature. It can be used to measure a number of characteristic properties of a sample such as fusion and crystallization. The result of a DSC experiment is a curve of heat flux versus temperature or versus time. There are two different conventions: exothermic reactions in the sample shown with a positive or negative peak, depending on the kind of technology used in the experiment. This curve can be used to calculate enthalpies of transition. This is done by integrating the peak corresponding to a given transition.

In other words the energy of the transition delta H is the area under the thermogram which is limited by the base line.

### Number Average Longest Crystal Dimension

Petrolatum crystals (also called crystallites) are elongated, the longest dimension is measured using microscopy.

Polarized light microscopy was performed using microscope Nikon Eclipse E 600 equipped with Linkam heating/cooling cell; 50× Plan objective was used to get micrographs.

### Petrolatum

Petrolatum is a flammable, semi-solid mixture of hydrocarbons, obtained from petroleum, and having a melting point ranging from a little below to a few degrees above 100° F. (37° C.). It is colourless or pale yellow (when not highly distilled), translucent and devoid of taste and smell when pure. It is insoluble in water. One source of petrolatum, for example, is Petrolatum Snow White® from Penreco.

In general, petrolatum (either the less structured petroleum jelly having lower elastic shear modulus G′, or the more structured PJ Snow White, or PJ yellow) is structured due to the presence of n-alkanes (paraffins) with the fraction of paraffins depending on the distillation process and source of crude oil. Structuring is defined by the modulus and yield stress of the component. Elastic shear modulus, measured at 30° C., frequency 1 rad/s and strain amplitude of 0.1%, is G′ =2000-3000 Pa for petroleum jelly Snow White, for example, and G′=200-300 for the less structured petroleum jelly liquid.

### Silicone Oil

Silicone oil can improve the sensory aesthetics (oiliness; tackiness) of petrolatum-based compositions. A typical silicone oil is polydimethyl siloxane where two methyl groups attach to the silicon atom to form (H₃C)₃SiO(CH₃)₂]Si(CH₃)₃ where n defines the length of the chain (n is typically >4). The upper limit of n is defined by the desirable viscosity of the silicone oil and may be 4 to 850, preferably 8 to 400.

Phenyl groups may be attached instead of methyl and would form, for example, phenyltris(trimethylsiloxy)silane (one phenyl group), tetraphenyl-dimethyldisiloxane (4 phenyl groups), trimethyl pentaphenyltrisiloxane (5 phenyl groups). Alkyl groups generally, for example, or a combination of phenyl and other alkyl groups may be grafted to the silicone backbone.

Examples of silicone oils which are sold as commercial products include DC-704®, DC 556 and PH-1555 HRI cosmetic grade silicone fluids sold by Dow Corning. Preferred oils include DC704 or DC556.

Preferably the cosmetic composition according to the invention contains less than 10% silicone oil.

In another preferred embodiment, the silicone oil/petrolatum ratio of the cosmetic composition is less than 10%, preferably less than 5%.

### Fatty Acids

Fatty acids according to the invention are C₁₂ to C₁₈ acids wherein ratios of larger chain to C₁₂ fatty acid are noted as below:
C₁₄:C₁₂ ratio (e.g., myristate to lauric): 1.18:1 to 2.0:1
C₁₆:C₁₂ ratio (e.g., palmitic to lauric): 1.8:1 to 2.5:1
C₁₈:C₁₂ ratio (e.g., stearic to lauric): 1.5:1 to 2.3:1.

Preferably, the cosmetic composition of the invention contains less than 8%, preferably less than 6% fatty acid.

### Cosmetic Compositions

The petrolatum composition according to the invention may be formulated into aqueous or anhydrous cosmetic compositions (e.g., body or facial care compositions) containing the petrolatum compositions (hereinafter, “PC”), e.g., as part of the hydrophobic or fatty phase.

For example, most cosmetic preparations contain, at varying levels of concentration, a hydrophobic or fatty phase comprising a mixture of oil, a fat and/or wax. This is true, for example, for oil-in-water or water-in-oil emulsions, gels, oils for face and body care, milks and make-up products such as rouge or lipstick.

The PC of the present invention may represent 1 to 80%, for example, of the total weight of the cosmetic composition. In the case of oil-in-water emulsion or body care compositions, PC will more typically represent 1 to 30%, preferably 2 to 15%, more preferably 2 to 10% by weight of the cosmetic composition. Typically, water will represent 70 to 99% by weight preferably 80 to 90% by weight of such compositions.

As compositions have less and less water, PC may reach from 50 to 80% by weight of the cosmetic. In a typical facial care application, PC would represent 40 to 70% by weight, preferably 45 to 65% by weight of the cosmetic composition.

Other components which can be used in the hydrophobic or fatty phase of a cosmetic composition are vegetable or animal oils, synthetic oils, fats and/or wax.

Among vegetable or animal oils which may be used include almond oil, avocado oil, olive oil, jujube oil, sesame oil, soybean oil, colza oil, squalene, lanolin and derivatives of any of the above.

Among synthetic oils may be used are ethyl and isopropyl palmitate, alkyl myristates (isopropyl, butyl or cetyl myristate), triglycerides of octanoic or decanoic acid, cetyl ricinoleate, stearyl octanoate, hydrogenated polyisobutene, etc.

Among waxes which may be used are included carnauba wax, beeswax, ozokerite, candelilla wax, Montan wax and microcrystalline waxes.

The hydrophobic phase can also contain small amounts of fatty alcohol (e.g., typically 0.1 to 3% by weight, preferably 0.2 to 1% by weight). These include long chain alcohols such as cetylic alcohol, stearylic alcohol, myristic alcohol, hydrostearylic alcohol, oleic alcohol and the like. In addition, the fatty phase may contain certain polymers, for example, polyvinyl pyrrolidine, typically in about 0.1 to 0.5% by weight.

As indicated, total amount of hydrophobic phase, including PC, varies depending on whether cosmetic composition is mostly aqueous, somewhat aqueous or non-aqueous and may vary typically from 3 to 99% by weight of the cosmetic compositions.

As noted, cosmetic compositions may be mostly aqueous or mostly anhydrous. The compositions may be fluid emulsions, lotions or more substantial emulsions. They may be, for example, milks or softening creams, milk or creams for hand care, makeup removing creams or milks, foundation bases, sunscreen milks or creams, artificial tanning milks or creams, milks or creams against perspiration, shaving creams or foams.

When in the form of cream or milk, the cosmetic composition is typically in the form of water-in-oil or oil-in-water emulsion wherein hydrophobic or fatty phase (including predominantly, 80 to 100% PC) represents 4 to 60% by weight, the water represents 30 to 90% by weight and an emulsifying agent represents 0.5 to 20%, preferably 1 to 12% by weight of cosmetic emulsion.

Among emulsifying agents, non-limiting examples which may be used are as follows:

Fatty polyoxyethylene or polyglycerol alcohols, oxyethylene or non-oxyethylene alkyl sulfates, mixtures of at least one lanoate (e.g., magnesium, calcium, lithium, zinc or aluminum lanoate and hydrogenated lanoline and/or lanoline alcohol, esters of fatty acids and polyols such as glycerol or propylene glycol). Glycerol and propylene glycol are also functioning, for example, as humectants. Also can be used monoesters of fatty acids and polyoxyethylene sorbitan.

Cosmetic compositions may also include thickening agents and gellifying agents. These include, for example, magnesium and aluminum silicates; ether-vinylic/anhydride maleic copolymers (e.g., polymer sold as “Viscofas”®); carboxyvinylic polymers such as those sold under the name Carbopol®; or gels of organically modified montmorillonite and neutral oil such as for example the product Miglyol gel®.

In addition, the cosmetic composition may comprise various other components, typically at levels of 0.1 to 3% by weight including coloring agents, perfumes, preserving agents, chelators, UV filters, pigments, pearlizing agents, mineral or organic fillers and vitamins.

### BRIEF DESCRIPTION OF THE INVENTION

It is a first object of the present invention to provide a petrolatum composition comprising:
(1) 50 to 99.95% by weight petrolatum, preferably at least 95%;
(2) greater than 0.05% by weight, of a copolymer bearing greater than 70% long chain alkyl.
   said composition having an enthalpy of between 30 and 65, preferably 40 and 60, even more preferably 45 and 55 J/g,
   the petrolatum number average longest crystal dimension being less than 10 μm, preferably less than 5 μm, more preferably less than 1 μm.

Preferably the petrolatum composition has a copolymer/petrolatum weight ratio of less than 1:100, more preferably less than 1:200.

Preferably petrolatum and copolymer represent together at least 95%, preferably at least 99%, more preferably at least 99.9% of the total weight of the petrolatum composition.

Preferably, the copolymer comprises vinyl amide monomer and long chain alkyl are α-olefins having chain length C₁₂ or greater.

Preferably also, the copolymer bears at least 75% long chain alkyl.

Preferably also the long chain alkyl are C₁₂ to C₃₀ alkyl, more preferably C₁₈ to C₂₄ alkyl. Copolymers of vinyl amide monomers, especially cyclic vinyl amide monomers (e.g., vinylpyrrolidone) and long chain (e.g., C₁₂⁻C₃₀) α-olefins (e.g., eicosane) are preferred.

It is a second object of the present invention to provide a process for manufacturing a petrolatum composition wherein the copolymer is added to the petrolatum, the petrolatum-copolymer blend is heated (65-75° C.) and cooled to room temperature.

In a preferred embodiment of the invention, petrolatum and copolymer are heated to a temperature of between 65 and 75 deg C before being added together.

In another preferred embodiment of the invention, petrolatum and copolymer are first added together and then heated to a temperature of between 65 and 75 deg C.

The petrolatum/copolymer composition is then combined with the other ingredients to produce a cosmetic composition.

A third preferred object of the invention is a cosmetic composition comprising 1 to 80% of a petrolatum composition according to the invention.

These and other aspects, features and advantages will become apparent to those of ordinary skill in the art from a reading of the following detailed description and the appended claims. For the avoidance of doubt, any feature of one aspect of the present invention may be utilized in any other aspect of the invention. It is noted that the examples given in the description below are intended to clarify the invention and are not intended to limit the invention to those examples per se. Other than in the experimental examples, or where otherwise indicated, all numbers expressing quantities of ingredients or reaction conditions used herein are to be understood as modified in all instances by the term “about”. Similarly, all percentages are weight/weight percentages of the total composition unless otherwise indicated. Numerical ranges expressed in the format “from x to y” are understood to include x and y. When for a specific feature multiple preferred ranges are described in the format “from x to y”, it is understood that all ranges combining the different endpoints are also contemplated. Where the term “comprising” is used in the specification or claims, it is not intended to exclude any terms, steps or features not specifically recited. All temperatures are in degrees Celsius (° C.) unless specified otherwise. All measurements are in SI units unless specified otherwise. All documents cited are—in relevant part—incorporated herein by reference.

### Examples of Petrolatum Compositions

Different types of petrolata were combined with a copolymer, the results are summarised in the following table.

Ganex V220 (a vinyl pyrrollidone/eicosene copolymer available from ISP International Speciality Products) was added to petrolatum and then the whole composition was heated to 65 deg C. for a period of at least 30 minutes, before being allowed to cool down to ambient temperature.

| | | Longest crystal |
|---|---|---|
| Type of petrolatum | Enthalpy, J/g | dimension |
| PJ Yellow G2212 (Control) | 51.7 | <40 |
| PJ White (2007) (Control) | 54.3 | <40 |
| PJ White (2007 + 0.1% Ganex V-220) | 49.1 | <5 |
| PJ G2212 + Ganex V220 | | |
| 0.1% | 46.0 | <5 |
| 0.15% | 47.8 | <5 |
| 0.2% | 47.2 | <5 |

The results show how adding a copolymer modifies the crystal size without significantly modifying the total crystal mass (as shown by the enthalpy). This results in an improved spreadability, hence better occlusion properties for cosmetic compositions,

Examples Of Cosmetic Compositions

Various cosmetic compositions were produced with petrolatum compositions contain 0.1% Ganex V220.

### EXAMPLE 1

### Skin Care Lotion

| | Chemical | % w/w |
|---|---|---|
| Phase A | Water | 78.05 |
| Phase B | Disodium EDTA | 0.05 |
| | Methylparaben | 0.21 |
| | Glycerin | 5 |
| | Triethanolamine | 1.44 |
| Phase C | Stearic acid | 4.57 |
| | Glycol stearate and stearamide AMP | 2.7 |
| | Glyceryl stearate | 1.27 |
| | Cetyl alcohol | 0.73 |
| | Petrolatum Yellow G2212 | 5.46 |
| | Ganex V220 | 0.0055 |
| | Propylparaben nf | 0.12 |
| Phase D | Phenoxyethanol | 0.4 |
| Final pH = 7.53 | | |
| Total | | 100 |

### EXAMPLE 2

### Skin Care Lotion

| | Chemical | % w/w |
|---|---|---|
| Phase A | Water | 48.52 |
| Phase B | Disodium EDTA | 0.05 |
| | Methylparaben | 0.21 |
| | Glycerin | 5 |
| | Triethanolamine | 1.44 |
| Phase C | Stearic acid | 4.57 |
| | Glycol stearate and stearamide AMP | 2.7 |
| | Glyceryl stearate | 1.26 |
| | Isopropyl Palmitate | 5 |
| | Cetyl alcohol | 0.72 |
| | Petrolatum Yellow G2212 | 30 |
| | Ganex V220 | 0.03 |
| | Propylparaben nf | 0.1 |
| Phase D | Phenoxyethanol | 0.4 |
| Final pH = 7.53 | | |
| total | | 100 |

### EXAMPLE 3

### Skin Lotions

| | A | B | C |
|---|---|---|---|
| Ingredients: | w/w % | w/w % | w/w % |
| DI Water | 86.060 | 84.060 | 57.030 |
| Phenoxyethanol | 0.400 | 0.400 | 0.400 |
| Disodium EDTA | 0.050 | 0.050 | 0.050 |
| Glycerine PF | 10.000 | 10.000 | 10.000 |
| Aristoflex | 0.690 | 0.690 | 0.690 |
| Methylparaben | 0.200 | 0.200 | 0.200 |
| Propylparaben | 0.100 | 0.100 | 0.100 |
| Tween 40 | 1.500 | 1.500 | 1.500 |
| Petroleum jelly modified | 1.000 | 3.000 | 30.000 |
| Ganex V220 | 0.001 | 0.003 | 0.030 |

| | E |
|---|---|
| Ingredients: | w/w % |
| DI Water | 38.120 |
| Agarose 3% shear gel | 12.700 |
| Disodium EDTA | 0.050 |
| Phenoxyethanol | 0.400 |
| Tween 40 (polyoxyethylene (20) | 2.000 |
| sorbitan monopalmitate) | |
| Methylparaben | 0.200 |
| Propylparaben | 0.100 |
| DC 9041 elastomer | 13.700 |
| DC 9045 | 12.025 |
| Dimethicone 50 cts | 2.620 |
| Dimethicone 5 cts | 1.000 |
| DC 245 | 5.025 |
| Petroleum jelly modified | 1.200 |
| Ultrez 21 | 0.260 |
| Glycerine PF | 7.500 |
| Tapioca starch | 2.700 |
| NaOH (10%) | 0.400 |

### EXAMPLE 4

### Skin Cleansers

| | A | B | C |
|---|---|---|---|
| Example | w/w % | w/w % | w/w % |
| Dove noodle (HEBE) | 14 | 2 | 0 |
| Cocamidopropyl betaine | 3.5 | 4 | 3 |
| Na laureth(1EO) sulfate | 5.8 | 2.5 | 8 |
| Na cocoyl glycinate | 0 | 3.5 | 0 |
| Lauric acid | 3 | 1.5 | 0 |
| Pure Gel B990 starch | 0 | 3.5 | 0 |
| Carbopol SF1 | 0 | 0 | 1.2 |
| D4051 cationic guar | 0.2 | 0.2 | 0.1 |
| Glycerin | 3 | 6 | 2 |
| Petroleum jelly modified | 5 | 15 | 30 |
| Perfume | 1 | 1 | 1 |
| Water | 64.5 | 60.8 | 54.7 |

1. A composition comprising:
i) 50 to 99.95% by weight petrolatum;
greater than 0.05% by weight of a copolymer bearing greater than 70% long chain alkyl.
said composition having an enthalpy of between 30 and 65 preferably 40 and 60, even more preferably 45 and 55 J/g,
the petrolatum number average longest crystal dimension being less than 10 μm, preferably less than 5 μm, more preferably less than 1 μm,
wherein the copolymer comprises vinyl amide monomer and long chain alkyl are α-olefins having chain length C₁₈₋₂₄.

2. (canceled)

2. A composition according to claim 1 wherein copolymers are copolymers of vinylpyrrilidone and long chain alkyl are C₁₈ to C₂₄ alpha olefins.

3. A process for producing a composition according to claim 1 wherein petrolatum and copolymer are added together and heated to a temperature of between 65 and 75 deg C. before being cooled down to a temperature of between 22 and 27 deg C.

4. A process according to claim 3 wherein petrolatum and copolymer are heated to a temperature of between 65 and 75 deg C. before being added together.

5. A process according to claim 3 wherein petrolatum and copolymer are first added together and then heated to a temperature of between 65 and 75 deg C.

6. A cosmetic composition comprising 1 to 80% of the composition of claim 1.

7. A cosmetic composition according to claim 6. containing less than 10% silicone oil.

8. A cosmetic composition according to claim 6 having a (silicone oil/petrolatum) weight ratio of less than 1:10

9. A cosmetic composition according to claim 6 containing less than 8%, preferably less than 6% fatty acid.
